(19) United States
(12) Patent Application Publication
(10) Pub. No.: US 2013/0291879 A1
(43) Pub. Date: Nov. 7, 2013

(54) TESTICULAR SUPPORT BELT FOR IMPROVING DISORDERS OF MICTURITION IN MEN

(71) Applicant: Yasuo MATSUDA, Higashi-Osaka-Shi (JP)

(72) Inventor: Yasuo MATSUDA, Higashi-Osaka-Shi (JP)

(21) Appl. No.: 13/867,201

(22) Filed: Apr. 22, 2013

(30) Foreign Application Priority Data
May 1, 2012 (KR) 10-2012-0045972

(51) Int. Cl. A61F 2/00

(52) U.S. Cl. CPC A61F 2/0009
USPC 128/885

(57) ABSTRACT

A testicular support belt for improving disorders of micturition in men and also helping to improve their quality of life by preventing deterioration in male testicular function, activating testicular function by improving the status of the micturition disorder, and preventing prostatic disease, while at the same time improving voiding completion, thereby reducing phenomena associated with disorders of micturition such as sensations of residual urine and the phenomenon of dripping after passing urine. A testicular support belt for men to improve disorders of micturition, wherein a scrotal support pad is sewn onto the inside or outside of a tape-type elastic component, and the elastic component includes a woven elastic tape, and the scrotal support pad a woven band of cotton fabric.

### TECHNICAL FIELD

The present invention relates to a means of improving disorders of micturition, and more specifically relates to a scrotal support belt helpful for overcoming urinary dysfunction such as disorders of micturition, for example, dysuria due to aging or prostatic hypertrophy (called a “testicular support belt” in this invention).

### BACKGROUND OF THE INVENTION

The testicle is the reproductive organ that symbolizes masculinity, and anatomically it comprises three major cell types, namely, hepatoblast cells that differentiate into mature sperm, Sertoli cells that have the role of a support, and Leydig cells called the testosterone-secreting cells. One each is located in the right and left of the scrotum. The testicles are not only the site where sperm, which are reproductive cells, are produced under the influence of male hormones, but may also be said to be the source of power and the starting point for life. If testicular function weakens, this may be manifested in the form of a kind of male functional disorder, for example, micturition disorders. Disorders of micturition mean conditions involving anything other than normal voiding of urine. The medical causes for them are known to be diverse and include bladder disease, prostate disease, urethral disease, neurogenic conditions and so on.

Male reproductive function increases until 24 years of age, on average, but beyond 24, cell function begins to deteriorate, and reproductive function weakens at the same time. Consequently, the scrotum sags and the testicles droop. Later on, deterioration in testicular function, such as weakening of the testicular sphincter with age, leads to functional weakening of the urethral sphincter, the muscular structure that holds the urine in the bladder until voiding commences. The prostate, which is linked to adult disease particular to men, relaxes and the scrotum sags still further. This not only affects even ejaculatory function, but also means that force is not applied to the urethra even when straining to pass urine. Quality of life is affected by increasing sensations of residual urine or urinary frequency and increasing discomfort due to oliguria (hypouresis), dripping after voiding and the phenomenon of urinary incontinence.

The present inventors established that such disorders of micturition could be greatly improved by lifting and supporting the scrotum a little above the usual level while carrying on with life and thus devised the present invention. This is probably because lifting and supporting the testicles relieves pressure on the scrotum and also testicular sphincters such as the urethral sphincter and serves to strengthen the endurance of the urethral sphincter.

Inventions or proposals have been published recently based on the common knowledge that keeping the scrotum 2-3° C. lower than body temperature is helpful for overcoming sexual dysfunction. For example, Korean Patent No. 10-0916707 describes men's functional pants capable of holding the penis and scrotum separately. It is explained therein that holding the penis and scrotum separately in this way helps to improve ventilation, and that maintaining the temperature of the scrotum below that of body temperature is advantageous for resolving sexual dysfunction problems. Functional pants with a similar construction for purposes the same or similar to this are also described in Korean Laid-open Patent 2001-0044219, Korean Laid-open Utility Model No. 1999-04507, and Korean Registered Utility Model Nos. 0301274, 0226297, 0245023, 0261821, 0362204, 0368726 and so on. The items described in Korean Laid-open Utility Model No. 1998-068586 also concern single-use penis or scrotum pouches to hold the penis and scrotum separately.

Moreover, Korean Registered Utility Model No. 0415398 and Korean Laid-open Patent Nos. 2010-0129490 and 2007-0019078 each describe open-type pants or moisture-proof pants constructed so that the scrotum does not touch the crotch, with fabric or a band cut to a U- or Y-shape sewn onto a belt between the penis and scrotum and/or between the scrotum and crotch so as to expose the penis and scrotum.

However, the principal function of all the functional male pants common to all the patents and utility models mentioned above is as pants worn to separate the penis and scrotum.

That is to say, the preceding patent references were devised making separate penis pouches and scrotum pouches in male pants, so that during wear, the scrotum is held separately from the penis and the area between the penis and scrotum will not become smelly or sweaty. They were not inventions or proposals whose main aim was to slightly raise the scrotum and also the testicles upwards.

There have, to date, been no examples proposing testicular support belts for improving micturition disorders that have been successfully realized. The term “support belt” in the above “testicular support belts” denotes a “band”, that is, a belt-shaped means of support.

### OBJECT OF THE INVENTION

The objective of the present invention is to provide a men's testicular support belt for improving disorders of micturition, and serving also to improve quality of life, by preventing deterioration in male testicular function, activating testicular function by improving the state of the micturition disorder, and preventing prostatic disease while at the same time giving better completion of voiding, and so reducing the phenomena associated with micturition disorders, such as sensations of residual urine or the phenomenon of dripping after passing urine.

### SUMMARY OF THE INVENTION

The aforesaid objective of the present invention can be met by the means of the present invention as set out below.

Testicular support belt for improving disorders of micturition in men, characterized in that a scrotal support pad is sewn onto the inner face or outer face of a tape-type elastic component.

The testicular support belt for improving disorders of micturition in men according to the invention, the elastic component is a woven elastic tape and the scrotal support pad is a woven band of cotton fabric.

Testicular support belt for improving disorders of micturition in men, according to the invention, the elastic component is linear or annular, and the scrotal support pad is rectangular, oval or double trapezoidal in shape.

Testicular support belt for improving disorders of micturition in men, according to the invention, the width ratio of the elastic component and the scrotal support pad is a proportion enabling the testicles to be supported in a stable fashion.

Testicular support belt for improving disorders of micturition in men, according to the invention, the width ratio of the elastic component and the scrotal support pad is approximately from 1:1 to 1:1.5 and the length ratio from approximately 1:5 to 1:5.5.

Testicular support belt for improving disorders of micturition in men, according to the invention, close fitting of the elastic component during wear is strengthened with a tape-type auxiliary elastic component.

Men's pants provided with a testicular support belt for improving disorders of micturition in men, characterized in that the elastic component of a testicular support belt for improving disorders of micturition in men, according to the invention, is sewn, with or without being cut, onto the inner face or outer face of the rubber waistband at the back of ordinary men's pants.

### ADVANTAGES OF THE INVENTION

When men with disorders of micturition due to ageing or similar continuously wear a testicular support belt for improving disorders of micturition in accordance with the present invention, the scrotum is lifted above its usual low position. This has the advantage of preventing drooping of the scrotum and testicles, thereby preventing weakening of micturition function in men and protecting against all manner of diseases linked to micturition disorders such as prostate-related diseases. In addition, increasing the intensity of voiding has the advantage of decreasing sensations of residual urine or the phenomenon of dripping after passing urine in men. In other words, it is beneficial for disorders of micturition.

### BRIEF DESCRIPTION OF THE DRAWINGS

FIG. 1(a) is a plane view showing the basic structure of the present invention.

FIG. 1(b) is the rear view of FIG. 1(a).

From FIG. 2(a) to FIG. 2(b), there are sketched some procedures for making a product of the present invention.

FIG. 3 illustrates products of the present invention with different shapes.

FIG. 4 is a sketch from the left side illustrating how a testicular support belt of the present invention is worn.

FIG. 5(a) illustrates a testicular support belt of the present invention joined to ordinary men's boxer shorts.

FIG. 5(b) is a sketch of the situation in FIG. 5(a) seen from the right.

FIG. 6(a), FIG. 6(b) and FIG. 7 are each sketches of other embodiments of testicular support belts of the present invention.

### DETAILED DESCRIPTION OF THE DRAWINGS

The invention is described in more detail in accordance with the attached drawings.

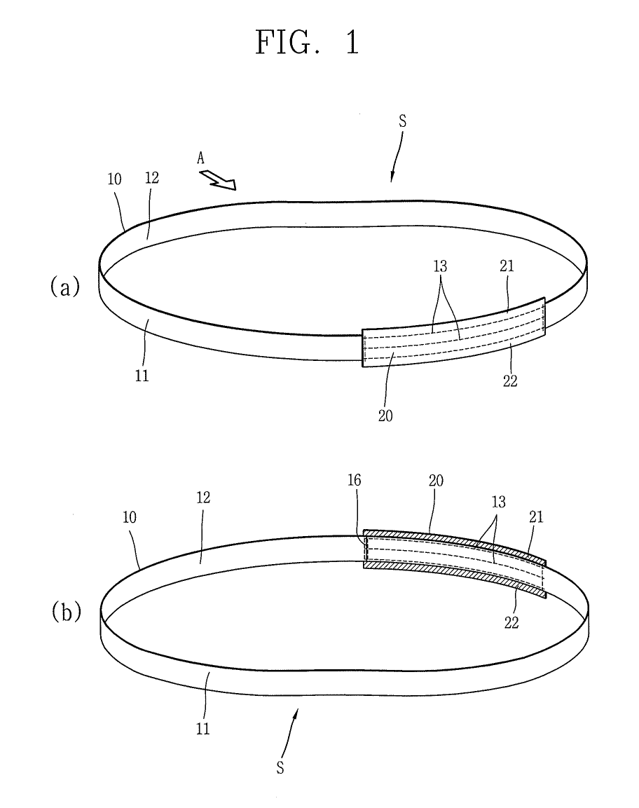

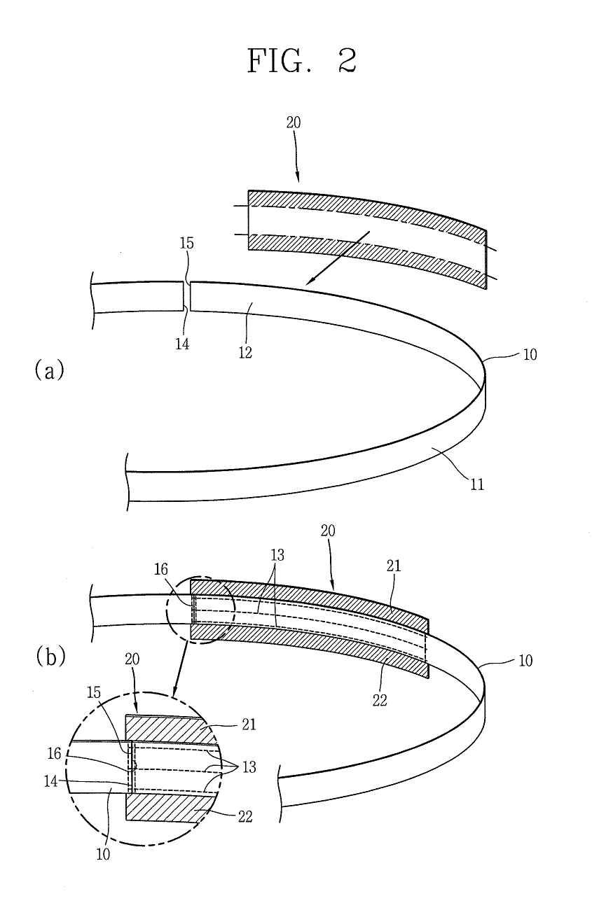

FIG. 1(a) is a sketch of a completed testicular support belt S of the present invention. The main structural components of the testicular support belt S can be classified into two parts, a tape-type elastic component (10) and a scrotal support pad (20) sewn at a set position on the outer face (11) or inner face (12) thereof. In the example illustrated, the scrotal support pad (20) has been sewn onto the outer face (11) of the elastic component (10). The term ‘sewn’ denotes the procedure of attaching by stitching.

The woven elastic tapes marketed for use in garments such as pants, pyjamas, sportswear (training wear) or trousers may also be used as the elastic component (10). Its dimensions do not differ either from those of woven elastic tapes in general. There are no restrictions on width, and a commercial product (e.g. 2.5-3.0 cm wide) fitted to the body as necessary, for example, cut to lengths ranging from about 65 to 80 cm may also be used. If the above elastic component (10) is made into a ring of the requisite dimensions in advance in accordance with the aims of the present invention, this may be used with ease as it is.

In general, the above-mentioned scrotal support pad (20) may be rectangular, oval or double trapezoidal in shape, or combinations of these, regardless of whether it is elastic or non-elastic. It is desirable for it to be, for example, a soft woven fabric band that will not have any effect such as chafing on the scrotum and surrounding skin, will allow good ventilation and will also absorb sweat well. Bands made of a soft cotton mesh material may also be used as necessary. However, there are no restrictions on the shapes or types of the materials used. The width ratio of the elastic component (10) and scrotal support pad (20) is set at about 1:1 to 1:1.5 and the length ratio at about 1:5 to 1:5.5, but they are not necessarily limited to these ranges.

When explaining the present invention, the expression “about” means that differences of a few millimeters but less than 1 centimeter are acceptable in the numerical values for width and length, in accordance with the specifications of materials for elastic components and materials for scrotal pouches available commercially.

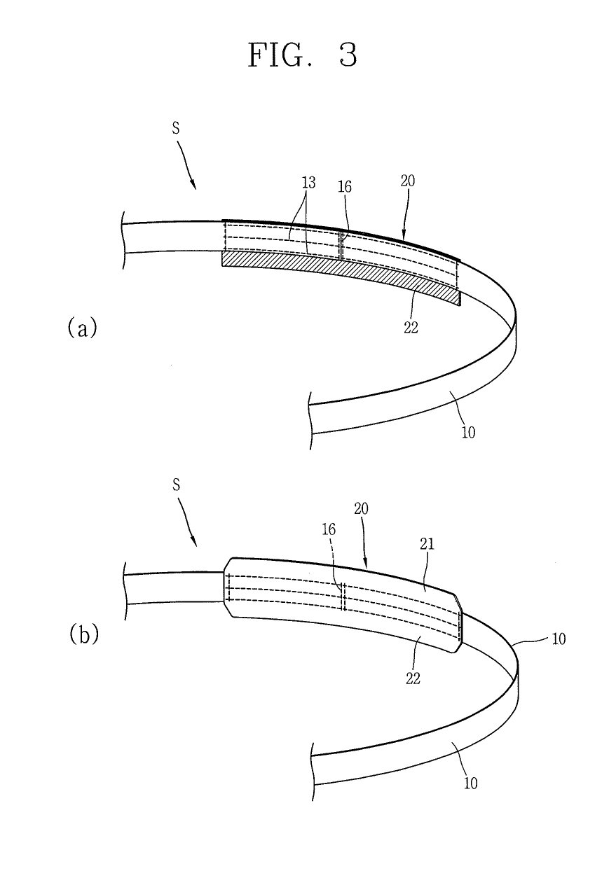

FIG. 1(b) is a sketch seen from the direction of the arrow A in FIG. 1(a). It shows an example with a rectangular scrotal support pad (20) sewn onto the outer face (11) of the elastic component (10) and illustrates a situation in which the ratio of the width of the elastic piece (10) and width of the scrotal support pad (20) is 1:1.5. In FIG. 1(b), an upper wing (21) and lower wing (22) of the scrotal support pad (20) are created with mutually equivalent width from the respective upper and lower edges of the elastic component (10). As may be realized from the width ratio of the elastic component (10) and scrotal support pad (20) described above, there may also be situations in which no upper and/or lower wings are formed. Moreover, the scrotal support pad (20) may also be sewn on so as to give either just an upper or lower wing, for example, only a lower wing (22), as shown in FIG. 3(a) described later. If the above elastic component (10) and scrotal support pad (20) have the same width as each other, then no upper or lower wing portions of the scrotal support pad (20) will be formed. Moreover, the scrotal support pad (20) may also be constructed to have an oval or double trapezoidal shape. Several lines of stitching (13) are produced as a result of the above sewing-on procedure. The detailed method of attachment for the above two components and the unexplained numeral 16 will be described later in connection with FIG. 2.

When implementing the present invention, a cotton elastic tape 2.4 cm wide was used as the elastic component (10) and a cotton band 3.5 cm wide×16 cm long as the scrotal support pad (20). However, as mentioned earlier, there is no specific range for the above dimensions and the size may be adjusted according to the standard physical dimensions of adult males.

FIG. 2 illustrates a method for sewing the above two components (10, 20) together to make a testicular support belt S of the present invention. That is to say, as sketched in FIG. 2(a), a testicular support belt S of the present invention can easily be made by sewing a scrotal support pad (20) with the requisite specification onto the outer face (11) of a tape-type elastic component (10) with the requisite specification. In this situation, if the elastic component (10) is a linear and not a circular band as illustrated, the two ends (14, 15) of the elastic component (10) are sewn closely together meeting at one end of the scrotal support pad (20) on the inside (the left side in FIG. 2(a)), forming a seam (16). The position of this seam (16) can be altered to facilitate stitching, and it should be arranged as illustrated so that the seam does not bulge in the long direction of the elastic component (10) to ensure that it does not come into contact with parts of the body such as the hips or flanks and irritate the skin. The seam (16) is positioned to the left edge of the scrotal support pad (20) in the example in FIG. 2(a) but may be set anywhere such as to the right edge or central part of the pad (20). If the elastic component (10) is not a straight band but a circular band, a seam (16) is not formed. The above seam (16) is not a structural requirement of the present invention, but if using an elastic component in the form of a straight band, arranging it in this way is also advantageous in product terms.

For convenience, FIG. 1(a), FIG. 1(b) and FIG. 2(a), FIG. 2(b) each illustrate an example in which rectangular scrotal support pad (20) is sewn onto the outer face (11) of the elastic component (10) to make a testicular support belt S. However, sewing a scrotal support pad (20) onto the inner face (12) of the elastic component (10) to make the testicular support belt S would not make any difference to its function for fulfilling the objective of the present invention. Scrotal support pads (20) that are rectangular, oval or double trapezoidal in shape, or combinations of these, may also be used.

FIG. 3(a) is a sketch showing a rectangular scrotal support pad (20) sewn onto a linear elastic component (10) so that, for example, only a lower wing (22) is formed. A join (16) for the two ends (14, 15) of the elastic component (10) is formed in the central portion of the face with the scrotal support pad (20) sewn onto the outer face (11). FIG. 3(b) is a sketch of an example using a double trapezoidal pad as the scrotal support pad (20) sewn onto the inner face (12) of a linear band type of elastic component (10). In FIG. 3(b), the parts with code numbers 21 and 22 displayed with the same width are the upper and lower wings of a double trapezoidal scrotal support pad (20) left over after covering the breadth of the elastic component (10). The seam (16) is created on the outside of the scrotal support pad (20) as illustrated in FIG. 2(b) and so cannot be seen in FIG. 3(b).

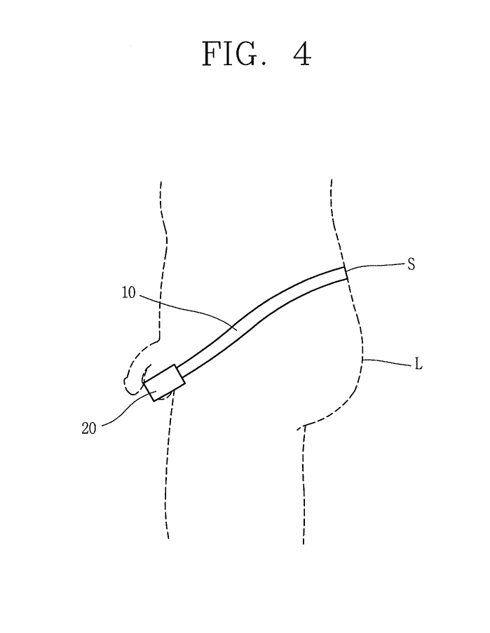

FIG. 4 is a sketch seen from the left showing how a testicular support S of the present invention is worn. The scrotum (not illustrated) is covered by the scrotal support pad (20) and due to the tensioning action of the elastic component (10) itself, the scrotum is lifted upwards to a degree capable of supporting the penis slightly horizontally or more. Underwear such as pants may be worn freely after the above testicular support S has been put on. The non-explained code letter L denotes the lower half of the human body.

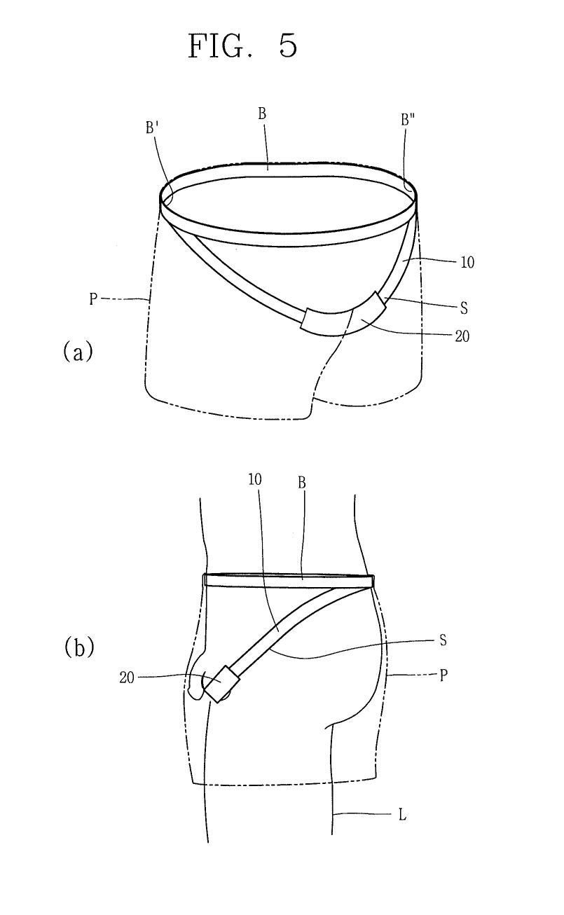

FIG. 5(a) is a sketch showing a testicular support S of the present invention sewn into the rubber waistband B of ordinary men's boxer short pants P. In this case, the central portion of the elastic component (10) of the testicular support S is cut on the side opposite the scrotal support pad (20) and it can be sewn like that inside the rubber waistband B of the pants P in the flank area (B′, B″), or onto the above waistband (B) at a suitable position further back on the hips from the flanks. At such times, the elastic component (10) may also be sewn directly over the rear portion of the rubber waistband B of the pants P without cutting. In this situation, the rear portion of the rubber waistband B of the pants P may feel somewhat thicker, but this poses no obstacle in terms of wearing, obviously, nor in terms of function. FIG. 5(b) is a see-through sketch from the right of the pants P in FIG. 5(a) being worn.

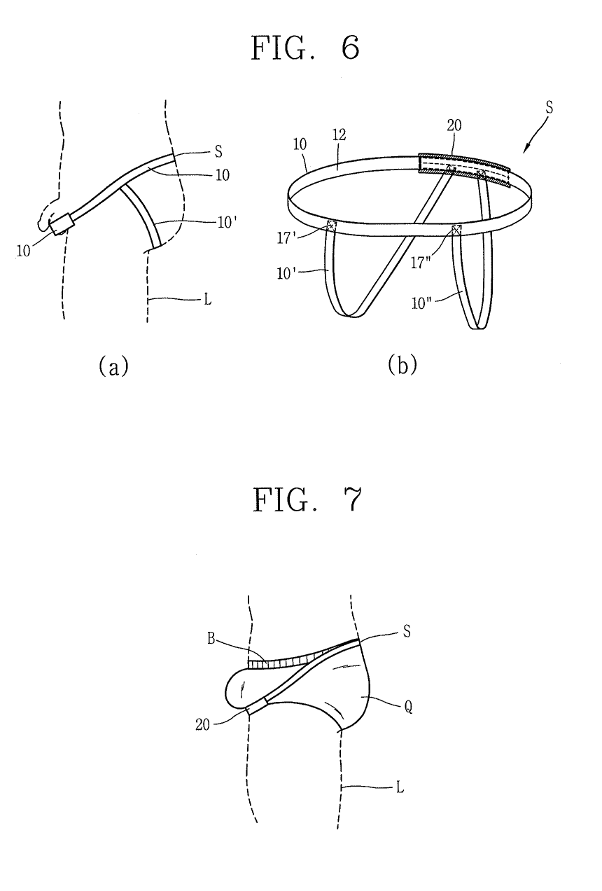

FIG. 6(a) and FIG. 6(b) are sketches that simply illustrate other possible embodiments of testicular support belts S of the present invention. For example, FIG. 6(a) shows an example using the testicular support belt S shown in FIG. 1, but in which the elastic component (10) is drawn down from the waist towards the scrotum and the scrotum (not illustrated) is lifted slightly. However, one end (17′, 17″) and the other end (18′, 18″) of two auxiliary pieces of elastic (10′, 10″) are each sewn at a suitable position on the elastic component (10) on the inner face (12) forward of the left and right hips, and to the scrotal support pad (20) (or they may be affixed in a freely removable fashion using buttons, hooks or Magic Tape), so that they pass through the crotch and are worn over the femoral region on both sides. Although not illustrated, the aforesaid other ends (18′, 18″) may also each be sewn on at any suitable position to the left and right of the elastic component (10) between the above attachment points (17′, 17″) and the scrotal support pad (20). The above system of sewing-on or attachment will be obvious to those skilled art. As the two auxiliary pieces of elastic (10′, 10″) are designed to pass through the crotch and sit at the crossover of the femoral region and buttocks, this can further improve close fitting of the elastic component (10). These auxiliary pieces of elastic (10′, 10″) are not absolutely required for the present invention. The auxiliary pieces of elastic (10′, 10″) may be made of the same material as the elastic component (10) but are not limited to this.

Furthermore, FIG. 7 illustrates an example in which a testicular support belt S of the present invention is worn outside ordinary men's short briefs Q. This gives a structure in which the scrotum (not illustrated) inside the short briefs Q is slightly lifted and supported. In view of the structure of the men's short briefs Q, the testicular support belt S of the present invention may be sewn directly onto the outer face at the back of the elastic waistband B of the above short brief Q, but it may also be sewn directly onto the inner face of the elastic waistband B of the short brief Q to bring the belt S inside the short brief.

It is obvious that persons skilled in the art would be able without great difficulty to make various changes and modifications within the scope of the invention to the construction of testicular support belts S, to the means of improving close fitting and practical methods for joining them to underwear such as pants other than as indicated in the above examples and embodiments.

What is claimed is:

1. A testicular support belt for improving disorders of micturition in men, wherein a scrotal support pad is sewn onto the inner face or outer face of a tape-type elastic component.

2. The testicular support belt for improving disorders of micturition in men according to claim 1, wherein the elastic component is a woven elastic tape and the scrotal support pad is a woven band of cotton fabric.

3. The testicular support belt for improving disorders of micturition in men according to claim 1, wherein the elastic component is linear or annular, and the scrotal support pad is rectangular, oval or double trapezoidal in shape, or a combination of these.

4. The testicular support belt for improving disorders of micturition in men according to claim 3, wherein the width ratio of the elastic component and the scrotal support pad is a proportion enabling the testicles to be supported in a stable fashion.

5. The testicular support belt for improving disorders of micturition in men according to claim 4, wherein the width ratio of the elastic component and the scrotal support pad is approximately from 1:1 to 1:1.5, and the length ratio from approximately 1:5 to 1:5.5.

6. The testicular support belt for improving disorders of micturition in men according to claim 1, wherein the close fitting of the elastic component is improved by using tape-type auxiliary elastic components.

7. The men's pants fitted with a testicular support belt for improving disorders of micturition in men, wherein the tape-type elastic component of a testicular support belt for improving disorders of micturition in men according to claim 1 is sewn, with or without being cut, onto the inner or outer face at the back of the rubber waistband of ordinary men's short briefs.
